(19) United States
(12) Patent Application Publication
(10) Pub. No.: US 2003/0144194 A1
(43) Pub. Date: Jul. 31, 2003

(54) Cancer treatment and prognosis

(76) Inventors: Anders Strom, Huddinge (SE); Jan-Ake Gustafsson, Stockholm (SE)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/204,644

(22) PCT Filed: Feb. 26, 2001
(86) PCT No.: PCT/EP01/02171

(30) Foreign Application Priority Data
Feb. 25, 2000 (GB) 0004568.2
Jul. 28, 2000 (GB) 0018587.6
Sep. 1, 2000 (GB) 0021508.7

(51) Int. Cl. A61K 38/17; A61K 31/137; A61K 31/452

(52) U.S. Cl. 514/12; 514/649; 514/651; 514/320

(57) ABSTRACT

This invention relates to methods of cancer treatment and prognosis and in particular to such methods involving the HES-1 protein.

This invention relates to methods of cancer treatment and prognosis and in particular to such methods involving the use or detection of the HES-1 protein.

Breast cancer is thought to occur as a result of estradiol stimulated proliferation of preinitiated epithelial clones (A. T. Ferguson, N. E. Davidson, Crit Rev Oncog 8, 29-46 (1997)). However, only a few of the downstream target genes for 17β-estradiol which mediate the effects on cellular proliferation, are known. We show that hairy and enhancer of split HES-1 (J. N. Feder, et al Mol Cell Biol 13, 105-13 (1993); A. Strom el al, Oncogene 19, 5951-5953 (2000) a basic helix-loop-helix (bHLH) factor, is a target of estradiol signalling. The HES-1 factor has been shown to repress neuronal differentiation when expressed ectopicly in the mammalian central nervous system (CNS) or chick retinal cells during development (M. Ishibashi et al, EMBO J 13, 1799-805 (1994); K Tomita et al, Neuron 16, 723-34 (1996)). HES-1 involvement in neuronal differentiation has also been shown in other systems such as the rat cell line PC12 where exogenous expression delays differentiation induced by NGF (A. Strom, et al, Genes Dev 11, 3168-81 (1997)) and also in embryonic day-17 hippocampal neurons in culture where down-regulation of endogenous HES-1 has been shown to be critical for neuronal differentiation (P. Castella, et al, J Neurosci Res. 56, 229-240 (1999)).

During development, HES-1 is an effector of Notch and therefore reacts to the stimulus produced as a result of Notch signalling. There are 4 mammalian forms of Notch receptors, and the corresponding ligands in vertebrates are Delta 1, Delta 3, Jagged 1 and Jagged 2. Notch receptors are transmembrane proteins, comprising both extra-cellular and cytosolic domains, which are involved in the control of the cellular response to developmental cues which specify cell fate (S. Artavanis-Tsakonas et al, Science 284, 770-6 (1999)).

In the fruit fly Drosophila, the HES-1 homologue hairy recruits the co-repressor groucho, and has been shown to repress transcription of genes involved in determining neuronal fate (A. L Fisher, M. Caudy, Genes Dev 12, 1931-40 (1998)). The mammalian homologues to groucho are the transducin-like enhancers of split (TLE) proteins 1-4 (S. Stifani, et al, Nat Genet 2, 119-27 (1992)) which have individually different tissue distributions where TLE 1 is the most widely expressed form.

HES-1 is found, in addition to the nervous system, in epithelial cells from lung, kidney, intestine (Y. Sasai, et al., Genes Dev 6, 2620-34 (1992)), in the pancreas, where notch signalling controls cellular differentiation (A. Apelqvist, et al Nature 400, 877-81 (1999)) and in T-cell precursors, where a lack of HES-1 expression leads to a significant reduction in T-cell numbers. (K. Tomita, et al., Genes Dev 13, 1203-10 (1999)). This implies that HES-1 plays an important role in the repression of cellular differentiation and continuation of cell proliferation. The same result can be seen in mice where the activity of HES-1 has been down regulated resulting in premature neurogenesis and neuronal differentiation (M. Ishabashi, et al., Genes Dev 9, 3136-48 (1995)). Recently HES-1 has been shown to be regulated by pref-1 expression during thymocyte development (M. Kaneta, et al., J Immunol 164, 256-64 (2000)) Pref-1 is a delta-like cell surface transmembrane protein, which in contrast to other delta-family proteins, lacks the DSL motif. It is proposed that the DSL motif is important for interaction with the Notch-family of cell surface, transmembrane receptors. This suggests that HES-1 may be open to regulation by mechanisms other than Notch signalling. HES-1 is also expressed in non-small cell lung cancer cells but is absent in small cell lung cancer cells (H. Chen. et al., Proc. Natl. Acad. Sci USA 94, 5355-60 (1997)). Small cell lung cancer is one of the most severe forms of lung cancer and proliferates and metastatizes very rapidly. This may indicate that a lack of HES-1 expression correlates to a more aggressive form of cancer.

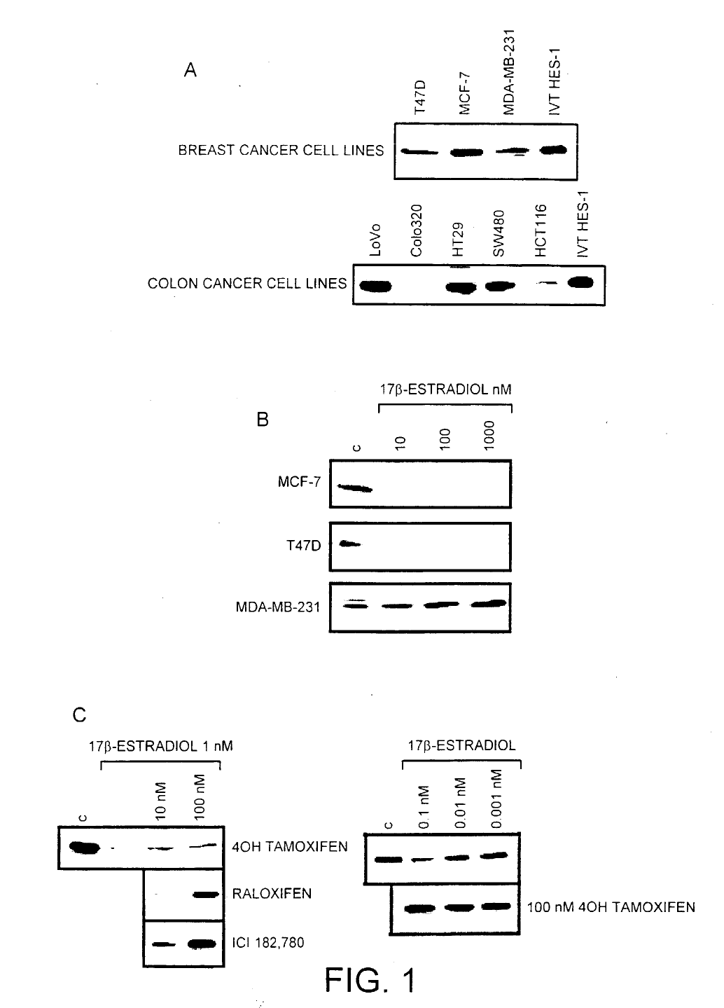

We investigated expression of HES-1 in epithelial cell types such as breast and colon cancer cell lines using antibodies to HES-1. Expression was found in all three breast cancer cell lines and was high in three out of five colon cancer cell lines. The expression level was found to vary somewhat between the different breast cancer cell lines tested. The highest levels of HES-1 expression were found in cell line MCF-7 followed by T47D and MDA-MB-231. In the case of the colon cancer cell lines tested, expression of HES-1 was highest in LoVo followed by HT29, SW480, HCT116 and Colo320 respectively. In the case of Colo320, HES-1 expression was found to be almost undetectable (FIG. 1A).

Our findings indicate that 17β-estradiol acts to suppress the expressed protein levels of HES-1 in T47D and MCF-7 breast cancer cells. The lack of 17β-estradiol-mediated suppression of HES-1 in MDA-MB-231 breast cancer cells is an indication that the estrogen receptor α (ERα) is needed for this regulation because these cells lack ERα (A. Friedl, V. C. Jordan, Eur J Cancer 10, 1559-64 (1994) (FIG. 1B). The antiestrogens 4OH tamoxifen. raloxifen and ICI 182,780 prevented 17β-estradiol-mediated down regulation of HES-1 protein. 4OH tamoxifen was less potent than raloxifen and ICI 182,780 in restoring HES-1 expression (FIG. 1C). The effects on HES-1 expression of antiestrogens further support involvement of the estrogen receptor in HES-1 regulation by 17β-estradiol.

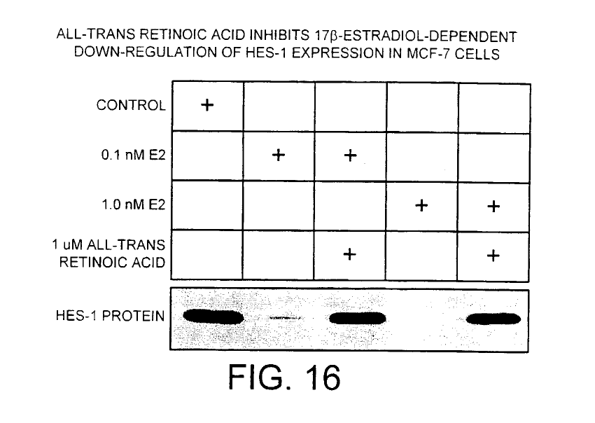

It has also been found that all-trans retinoic acid inhibits 17β-estradiol-dependent down regulation of HES-1 expression in MCF-7 cells (FIG. 16). This is an important finding since retinoic acid and retinoids have been shown to enhance the effect of tamoxifen on proliferation in breast cancer.

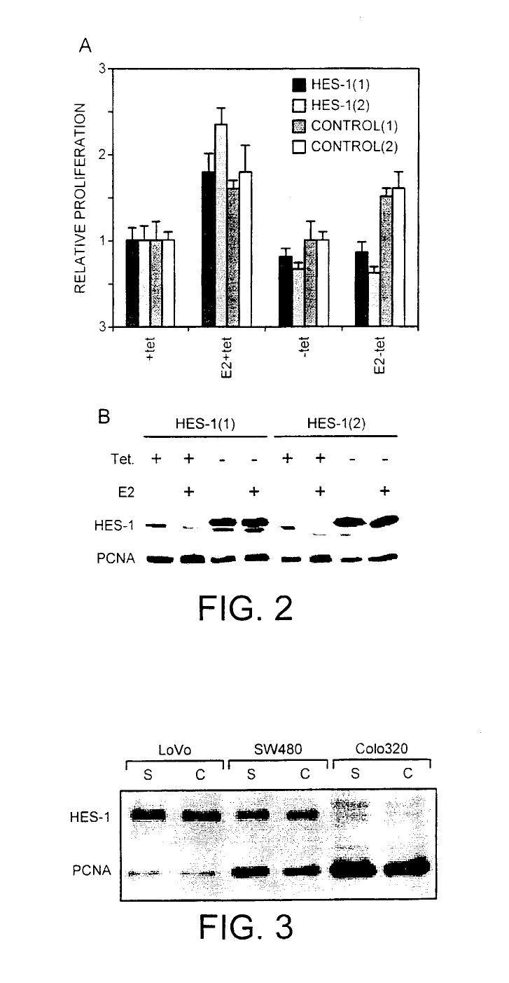

We also used “tet-off” T47D breast cancer cells stably transfected with a FLAG-HES-1 tetracycline regulated expression vector, to study if induced expression of exogenous HES-1 could affect proliferation. When cells were grown in the presence of tetracycline and thus without induced HES-1 expression, a normal two-fold stimulation of proliferation by 17β-estradiol treatment was observed. However, in the absence of tetracycline (i.e.with induced HES-1 expression) no 17β-estradiol stimulated proliferation was obtained. This strongly suggests that HES-1 protein levels need to be down-regulated for proliferation to increase following treatment with 17β-estradiol (FIG. 2A). The proliferation marker, proliferating cell nuclear antigen (PCNA) was upregulated twofold by estrogen in the absence of exogenous FLAG-HES-1 expression. In contrast, no regulation was found in the presence of exogenous FLAG-HES-1 (FIG. 2B). This finding suggests that PCNA is a target gene for HES-1. In the absence of expression of exogenous HES-1 17β-estradiol regulation of endogenous HES-1 was observed (FIG. 2B). This confirms that the cells have not lost their ability to respond to 17β-estradiol by clonal selection, and that the mechanism of stimulated proliferation is likely to be mediated by HES-1. Two control cell lines which were stably transfected with the tetracycline-regulated expression vector lacking FLAG-HES-1 exhibited 17β-estradiol-stimulated proliferation, confirming that expression of the VP16 fused transactivator (a component of the tetracycline expression system) did not inhibit 17β-estradiol-stimulated proliferation to any great extent.

Colon cancer cell lines were also used to investigate whether there is a correlation between HES-1 expression and the marker of proliferation (PCNA) in another epithelial cell line to breast cancer cells. Western blotting with antibodies against HES-1 and PCNA showed an inverse correlation between the protein levels of the two factors. This indicates that a reduction in HES-1 expression allows higher expression of PCNA (FIG. 3).

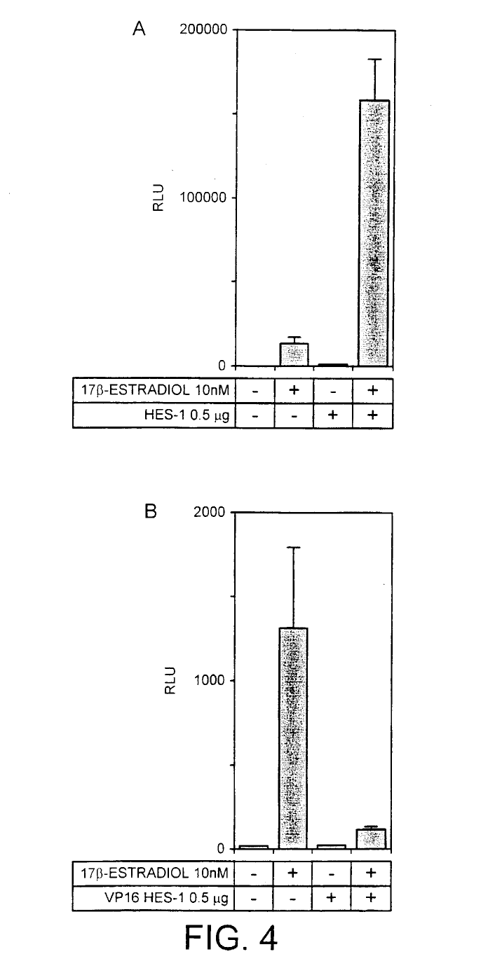

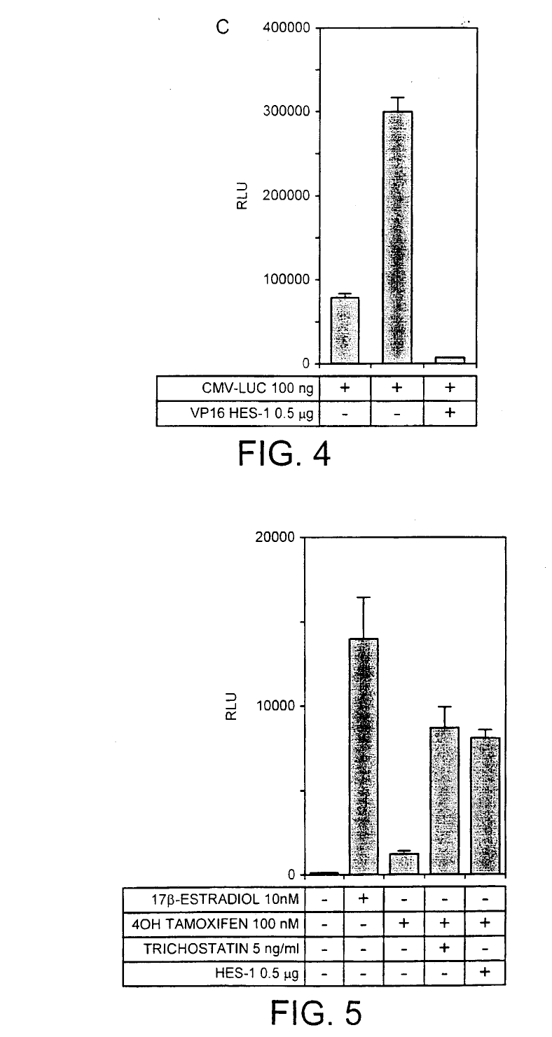

Our findings also indicate that expression of exogenous HES-1 increased the 17β-estradiol response of a 17β-estradiol responsive promoter (3×ERE TATA LUC) construct transfected into the breast cancer cell line T47D (FIG. 4A). Since HES-1 is a well characterised repressor of transcription, and mediates this effect by binding to the class C elements CACGCG or CACGAG or the class B element CACGTG found upstream of promoter sequences (A. L. Fisher. M. Caudy, Genes Dev 12, 1931-40 (1998)) we transformed HES-1 into an activator by fusing a VP16 activation domain to the N-terminal part of the protein. The VP16 HES-1 fusion protein had the opposite effect to that which is characteristic of the native or wild type (w.t.) HES-1, inhibiting 17β-estradiol-induced expression from the described construct (FIG. 4B). This indicates that HES-1 transcriptionally down regulates a factor which either directly or indirectly affects estrogen receptor activity. The same result was obtained with respect to the transcriptional activity of the cytomegalovirus (CMV) promoter, as judged by transient transfection assays of CMV promoter-driven expression constructs (FIG. 4C). This suggests that the effect of HES-1 on transcription is not limited to involvement with the estrogen receptor but may have a more general action affecting several different promoters. A possible candidate factor for the inhibition of estrogen activity may be a histone deacetylase enzyme. Furthermore, histone deacetylase inhibitors such as trichostatin A and n-butyrate have been shown to induce CMV promoter activity. (L. D. Dion, et al., Virology 231, 201-9 (1997)).

Injection of antibodies against HDAC2 (Histone deacetylase 2) into MCF-7 breast cancer cells has been shown to transform 4OH tamoxifen into an effective agonist (R. M. Lavinsky, et al., Proc. Natl. Acad. Sci. USA 95, 2920-5 (1998)). We show here that HES-1, expressed together with estrogen receptor a and an estradiol responsive reporter ERE TATA LUC in COS-7 cells, also makes 4OH tamoxifen into an effective agonist. The same result has been obtained by treating cells with the histone deacetylase inhibitor-trichostatin A (FIG. 5). This finding indicates that HES-1 potentially regulates histone deacetylase activity. Thus, estrogen treatment of breast cancer cells should increase histone deacetylase activity as a consequence of down regulation of HES-1 protein. Furthermore, this finding is also in agreement with the finding by Pasqualini et al that 17β-estradiol treatment of MCF-7 breast cancer cells decreases histone acetylation (J. R. Pasqualini el al, Breast Cancer Res. Treat 14, 101-5 (1989)).

Histone deacetylase inhibitors have recently been shown to inhibit proliferation (Y B Kim, et al (1999) Oncogene 18, 2461-70) which suggests one possible mechanism whereby HES-1 could affect proliferation.

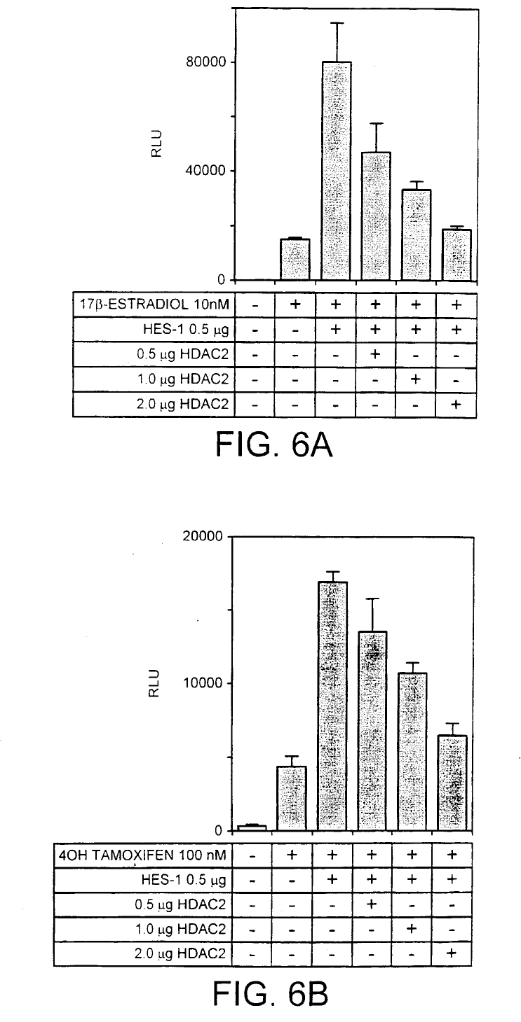

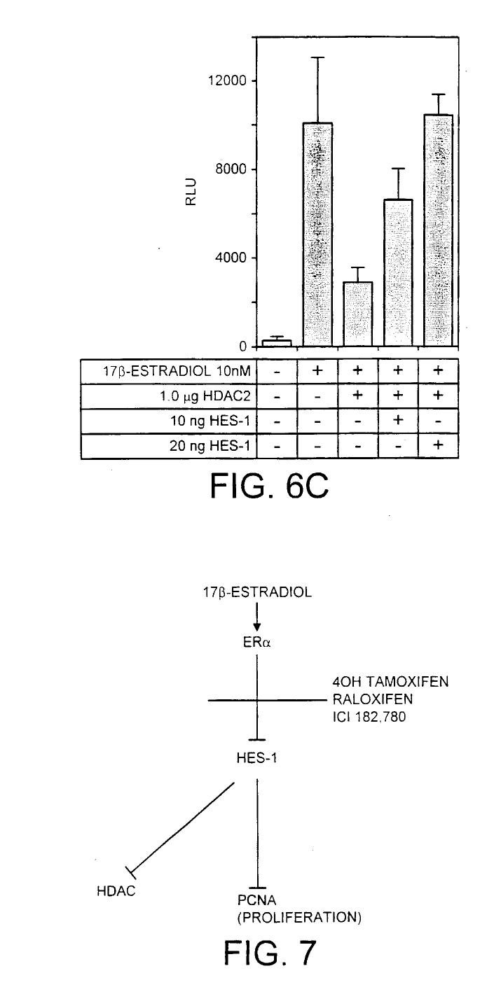

In order to investigate if regulation of histone deacetylase activity could be a possible mechanism for the HES-1 mediated increase of estrogen response on ERE dependent reporters, we tested if co-expression of HDAC2 could neutralise the HES-1 increased estrogen response. We found that transfected HDAC2 expression vector drastically reduced the increase of estrogen response caused by HES-1 expression in T47D cells (FIG. 6A). Furthermore, the same treatment reduced the increased agonistic response of 4OH tamoxifen caused by HES-1 expression (FIG. 6B). Finally, estrogen response in T47D cells which was decreased by HDAC2 expression could be rescued by co-transfection with HES-1 expression plasmid (FIG. 6C). These findings indicate that HDAC2 is a good candidate as either a direct or an indirect target gene for HES-1. At this stage however, it cannot be excluded that another histone deacetylase is regulated by HES-1 and is substituted for by HDAC2 in our experiments.

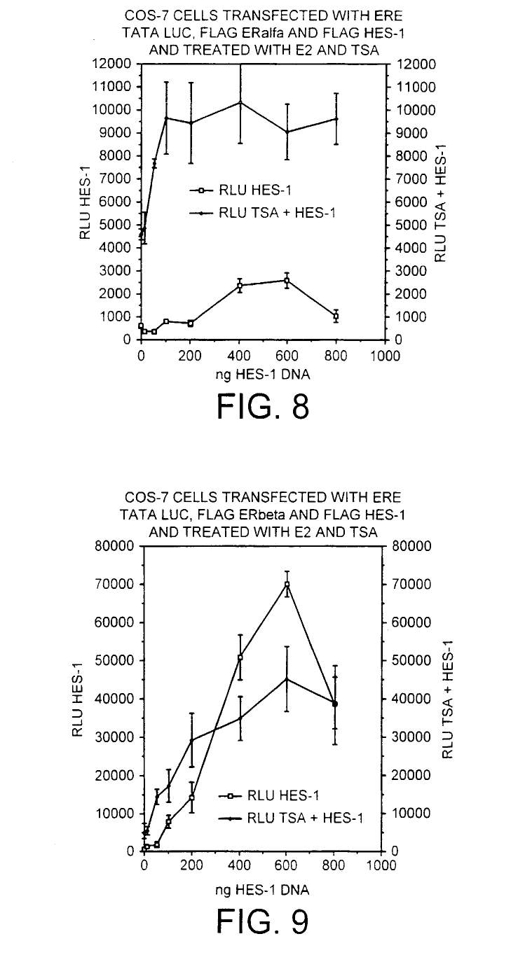

Our results indicate that the effect of HES-1 on the 17β-estradiol response differs between ERα and ERβ. Transient transfections of COS-7 cells using the ERE reporter vector ERE TATA LUC and expression vectors for ERα and HES-1 indicate that, in respect of ERα, HES-1 increases the response by only two to three fold at 400 ng transfected HES-1 expression plasmid. In contrast, under the same conditions ERβ shows a ten to twenty fold increase in response (FIGS. 8 and 9).

Furthermore, the addition of histone deacetylase inhibitor, trichostatin A (TSA) together with HES-1 provides a much larger response in the case of ERα. This indicates that HES-1 alone is not responsible for removal of ERα repression (FIG. 8). However, in the case of ERβ, the addition of TSA in combination with HES-1 made no significant difference (FIG. 9). These results indicate that HES-1 is probably regulating a histone deacetylase which is more important in ERβ activity than in ERα activity. This histone deacetylase is likely to be HDAC2.

Accordingly, if a specific inhibitor to the histone deacetylase regulated by HES-1 (presumably HDAC2) can be developed, ERβ activity could be increased relative to ERα activity through use of such an inhibitor. This inhibitor would have a similar proliferation inhibiting effect as that of HES-1 and would therefore be useful in the treatment of cancer. An anti-proliferative, breast cancer therapy strategy in accordance with the invention is aimed at inhibiting the activity of ERα. Such strategies operate via the mechanistic prevention of HES-1 downregulation, as expression of exogenous HES-1 prevents a 17β-estradiol mediated increase in cellular proliferation in T47D cells (FIG. 2A). A model of this regulation is shown in FIG. 7. Regulation of HES-1 either transcriptionally or post-translationally might be crucial in cells expressing the factor and for those cells to respond to proliferation inducing agents.

Our results suggest that HES-1 works not only as a regulator of differentiation as shown before but also by controlling proliferation. Perhaps the expression level of HES-1 determines the rate of cellular proliferation and also prevents differentiation in the nervous system. However, in epithelial cells HES-1 might have a more pronounced role as a regulator of proliferation in response to different cellular stimulators, which makes HES-1 interesting as a potential target for cancer treatment. The activity of HES-1 could therefore be useful to accurately control cell proliferation in respect of, cell number, timing of proliferation activation, proliferation speed as well as controlling cell size to expand the cell number at the right time with the right speed and to the right size. Our results suggest that HES-1 works not only as a regulator of differentiation as shown before but also by controlling proliferation. Perhaps the expression level of HES-1 determines the rate of cellular proliferation and also prevents differentiation in the nervous system. However, in epithelial cells HES-1 might have a more pronounced role as a regulator of proliferation in response to different cellular stimulators, which makes HES-1 interesting as a potential target for cancer treatment.

In the present specification, the term “HES-1” in relation to a protein or polypeptide embraces synthetic or artificial homologues of the wildtype or native protein, and variants including sequence variants, including insertion or deletion mutations or substitutions of amino acid residues for similar residues having similar characteristics. In relation to nucleotide sequences “HES-1” covers variations on the native sequence including insertions and deletion mutants and sequences which hybridise to the native sequence under stringent conditions and variants due to the degeneracy of the genetic code.

According to one aspect of the invention there is provided the use of HES-1 in the preparation of a medicament for the treatment of cancer. The HES-1 can be in the form of the protein or corresponding nucleotide preferably in a suitable vector. The HES-1 can be as defined above.

According to another aspect of the invention there is provided the use of an inhibitor to the histone deacetylase enzyme regulated by HES-1 in the preparation of a medicament for the treatment of cancer. An example of such an inhibitor is trichostatin A.

According to a further aspect of the invention there is provided the use of an inhibitor of ER activity in the preparation of a medicament for the reduction of cancer cell proliferation. Such inhibitors may be antiestrogens such as 4OH tamoxifen, raloxifen or ICI 182, 780.

The above uses may involve the upregulation of HES-1.

According to another aspect of the invention there is provided a method of reducing proliferation of cancer cells comprising increasing levels of HES-1 in those cells. HES-1 may also regulate histone deacetylase activity.

HES-1 levels may be increased for example by upregulation of genes expressing HES-1 in those cells by introduction of HES-1 protein or by introducing exogenous HES-1 expression in those cells through gene therapy. The effect of HES-1 on reduction of proliferation may be enhanced by the expression of engineered HES-1 which has improved properties compared to native or wildtype HES-1. The engineered HES-1 may be expressed by a gene which replaces the native HES-1 encoding gene or may complement that gene for example by exogenous expression of a gene for the engineered HES-1. The effect of HES-1 on proliferation may be enhanced by an antiestrogen.

The cancer cells may be selected from breast, colon, prostate and lung cancer cells. Breast and colon cells are preferred. The cancer cells may be in vivo or in vitro.

According to a further aspect of the invention, there is provided a method of monitoring cell proliferation by monitoring the expression of a marker such as PCNA or Ki67. Proliferation may be induced by estradiol.

According to another aspect of the invention, there is provided a method of prognosis with respect to the outcome of cancer in cancer cells in vitro or in vivo comprising establishing levels of HES-1 expression in those cells in combination with PCNA or Ki67 determination. HES-1 expression levels exhibited an inverse correlation with PCNA (FIG. 3). Protein levels differed markedly but RNA levels were the same indicating that this correlation is likely to result from a translation related effect such as HES-1-translation modulation. The cancer cells may be breast, colon or prostate cancer cells.

According to another aspect of the invention, there is provided a method of monitoring the effectiveness or progress of cancer in cancer cells in vitro or in vivo comprising determining the levels of HES-1 in those cells wherein lower levels of HES-1 are indicative of increased proliferation.

According to another aspect of the invention, there is provided a method of screening compounds, the method comprising detecting the effect of those compounds on HES-1. Preferably, the compound enhances the effects of HES-1, but compounds which decrease the effect of HES-1 may also be of interest. Preferably, the compound enhances the expression of HES-1. Preferred compounds will enhance HES-1 activity for example through increasing binding affinity.

The invention also provides a method of identifying compounds that regulate HES-1 gene expression, the method comprising contacting such compounds with an HES-1 gene or gene expression model. The compounds may be selected from compounds which function as a ligand to a nuclear receptor such as the retinoic acid receptor, the Vitamin D receptor, the estrogen receptor, aryihydrocarbon receptor (AhR), dioxin receptor or the TCDD receptor.

According to another aspect of the invention there is provided the use of an HES-1 nucleotide sequence or part thereof in the preparation of a medicament for gene therapy of cancer. There is also provided a method of gene therapy comprising supplying to cells a nucleotide sequence of HES-1 or part thereof. For example, the nucleotide sequence may comprise the entire HES-1 gene or may comprise a portion encoding a functional portion of HES-1. The function may be the reduction in proliferation of cancer cells. The nucleotide sequence may be supplied to the cells in a vector such as a plasmid or virus in accordance with conventional gene therapy techniques. Preferably, the HES-1 or a portion thereof is expressed in the cells whereby proliferation of those cells is reduced.

The HES-1 nucleotide sequence may be supplied in combination with another anti-cancer therapy. For example, it may be supplied with a conventional anti-cancer drug.

According to a still further aspect of the invention there is provided a pharmaceutical preparation comprising HES-1 protein or corresponding nucleotide either native or synthetic or in any pharmacologically effective variation thereof.

According to another aspect of the invention there is provided a pharmaceutical preparation comprising an inhibitor to the histone deacetylase regulated by HES-1.

Pharmaceutical compositions of this invention comprise any of the compounds of the present invention, and pharmaceutically acceptable salts thereof with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tnrsilicate, polyvinyl pyrrolidone, cellulose-based substances, polyethylene glycol, sodium carboxymethylcellulose, polyacrylates, waxes, polyethylene- polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. We prefer oral administration or administration by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrastemal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non- irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The invention also embraces modified forms of HES-1 such as constructs including HES-1.

The invention will now be described, by way of example only, with reference to the accompanying FIGS. 1 to 16 in which:

FIG. 1(A) is a Western blot of nuclear extracts from breast and colon cancer cell lines using an antibody to HES-1:100 μg of nuclear protein was used; WVT HES-1 is in vitro translation protein.

FIG. 1(B) Regulation of HES-1 by 17β-estradiol in breast cancer cell lines; 50 μg of nuclear extract protein was used;

FIG. 1(C) shows that 17β-estradiol regulation of HES-1 can be prevented by antagonists to the estrogen receptor; 50 μg of nuclear protein was used.

FIG. 2 illustrates a proliferation assay T47D cells stably transfected with tetracycline regulated FLAG HES-1 expression. (A) Relative proliferation of two different clones HES-1(1) and HES-1(2) with inducible FLAG HES-1 expression, and two control clones. Tetracycline at 1 μg/ml and 17β-estradiol at 10 nM were used when stated. Data represents an average of six independent measurements. (B) Western blot of endogenous HES-1 and FLAG HES-1 expression in HES-1(1) and HES-1(2) cell lines with antibody against HES-1. PCNA Western blot on same extracts 50 μg of nuclear protein was used;

FIG. 3. Western blot using nuclear extracts from colon cancer cell lines and antibodies to HES-1 and PCNA respectively. 50 μg of protein was loaded in each lane. Subconfluent (S) and confluent (C) cells respectively were used;

FIG. 4(A) Transfection of T47D cells with 3X ERE TATA LUC. 1 μg, 0.5 μg of pcDNA3, FLAG HES-1; treatment was with 10 nM 17β-estradiol. (B) Same as for A but pcDNA3, VP16 HES-1 was used instead of pcDNA3 FLAG HES-1 (C) Transfection of COS-7 cells with a CMV luciferase construct either together with wt HES-1 or VP16 HES-1 0.5 μg in each case;

FIG. 5. Transfection of COS-7 cells with 3X ERE TATA LUC 1 μg and 100 ng of pcDNA3 FLAG ERα. Treatment was with 10 nM 17,-estradiol, 100 nM 4OH tamoxifen and 5 ng/ml trichostatin A for 8 hrs; and

FIG. 6. (A) T47D cells transfected with 3X ERE TATA LUC 1 μg, pcDNA3 flag-HES-1 0.5 μg and pME18S HDAC2, 0.5, 1.0 respective 2.0 μg; treatment was with 10 nM 17β-estradiol for 24 h. (B) COS-7 cells transfected in the same way as (A) but 100 nM 4OH tamoxifen was used instead of 17β-estradiol (C) Bars 1 and 2, T47D cells transfected with the above stated reporter. Bars 3 and 4, in addition to reporter 1.0 μg pME18S HDAC2 and 10 respectively 20 ng of pcDNA3 FLAG HES-1.

FIG. 7. Model of 17β-estradiol regulation of HES-1 and downstream targets.

FIG. 8. Transient transfection of COS-7 cells using the ERE reporter vector ERE TATA LUC and expression vectors FLAG ERα and FLAG HES-1 and treated with E2 and the histone deacetylase inhibitor Trichostatin A (TSA).

FIG. 9. Transient transfection of COS-7 cells using the ERE reporter vector ERE TATA LUC and expression vectors FLAG ERG and FLAG HES-1 and treated with E2 and the histone deacetylase inhibitor Tricostatin A (TSA).

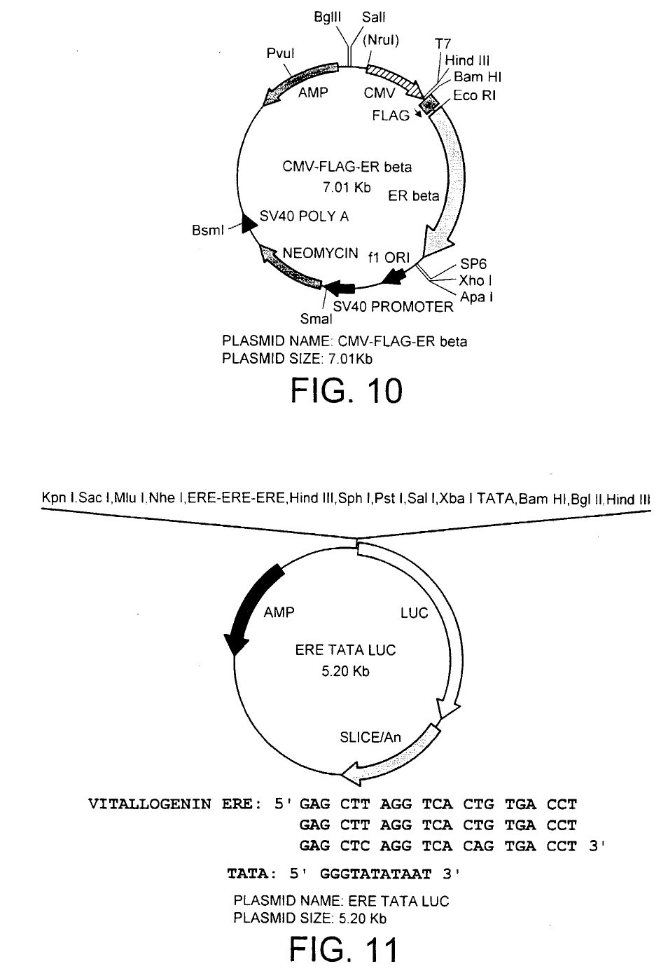

FIG. 10 is a schematic representation of plasmid CMV-FLAG ERG.

FIG. 11 is a schematic representation of plasmid ERE TATA LUC.

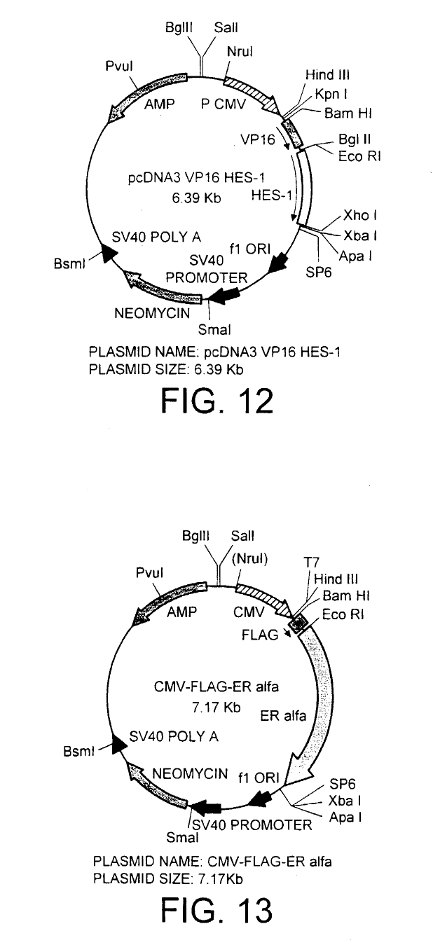

FIG. 12 is a schematic representation of plasmid pcDNA3 VP 16 HES-1.

FIG. 13 is a schematic representation of plasmid CMV-FLAG-ERα.

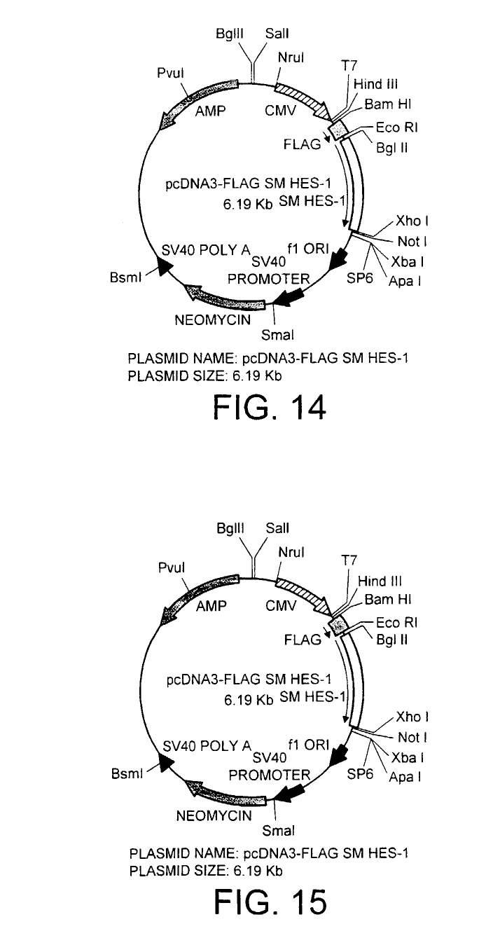

FIG. 14 is a schematic representation of plasmid pcDNA3-FLAG HES-1.

FIG. 15 is schematic representation of plasmid pcDNA3-FLAG SM HES-1.

FIG. 16 shows that all-trans retinoic acid inhibits 17β-estradiol-dependent down regulation of HES-1 expression in MCF-7 cells.

Materials and Methods

1) Expression Constructs

pcDNA3 FLAG HES-1 (P. Castella, J. Wagner, M. Caudy, J. Neurosci Res 56, 229-240 (1999)) was used for preparation of FLAG HES-1 which was subsequently cloned into the PBI-EGFP vector (Clontech). Human ERα was epitope tagged with the artificial “FLAG” epitope using PCR cloning. The VP16 activation domain was fused to the N-tenninus of rat HES-1.

2) Cell Culture, Stable Transfections

T47D cells were cultured in a 1:1 mixture of Hams F 12 and DME with 10% fetal bovine serum, MCF-7, MDA-MB-231 and COS-7 cells were cultured in DME with 10% fetal bovine serum. Before experiments with estrogen, the media were switched and phenol red free and DCC charcoal treated with serum were used. Culture conditions were set at 37° C., 5% CO₂ and 100% humidity. For creation of tetracycline inducible stably transfected clones the tet-regulated expression system from Bibco BRL was used. A puromycin resistance gene was cloned into pTet TAk. T47D cells were then transfected with lipofectin (GibcoBRL) according to the manufacturer's instructions. Transfectants were selected with puromycin at 1 μg/ml. One stably transfected clone which showed good tetracycline regulation of a transiently transfected plasmid containing a luciferase gene cloned after a tetracycline-regulated promoter was used for the next round of transfections of PBI-EGFP (Clontech) plasmids containing cDNA for HES-1. Transfectants were selected using G418 (Calbiochem) at 500 μg/ml. Cells lines stably transfected with tetracycline regulated expression constructs were grown in the presence of 1 μg/ml of tetracycline. At the time of induction the medium was removed and the cells were washed with PBS two times before adding new medium without tetracycline.

3) Nuclear Extracts

Nuclear extract and RNA preparation was carried out according to the protocol by Schreiber et al (E. Schreiber, et al., Nucleic Acids Res 17, 6419 (1989)) except that PBS was used instead of TBS and the following inhibitors were added: leupeptin at 1 μg/ml, PMSF at 0.2 mM, 100 pM Na₃VO₄, 1 mM NaPPi, 1 mM NaF, 100 μM Na₂MO₄ (all from Sigma). Nuclear extracts were prepared from cells grown to a confluency of 60%-70% on a 150 mm culture dish. Protein content was determined by Bradford assay (BioRad).

4) Production of HES-1 Antibody and Western Blot Analysis

Two peptides with identical sequence parts of HES-1 protein were synthesised and simultaneously injected into one rabbit (Genosys). The peptides were CMEKNSSS PVAATPASVNTTPDKPKTASEHR and CSGTSVGPNAVSPSSGSSLTADSMWRPWRN. For Western blots, 100 μg of nuclear extract was separated on a 12% SDS-PAGE. Western blot analysis was performed according to standard protocols (Harlow and Lane, E. Harlow, and D. Lane, Cold Spring Harbour Laboratory Press, Cold Spring Harbour, N.Y., (1988)). HES-1 antibody was used at a dilution of 1:1000. The PCNA (C-10) antibody was from Santa Cruz and used at 1:1000. Donkey, anti-rabbit and sheep anti-mouse IgG HRP conjugated antibody (Amersham) were used at 1:10000. Antibody binding was visualised using ECL, Super Signal (Pierce).

5) Proliferation Assay

A proliferation determination kit from BioThema (A. Lundin, Methods Enzymol 305, 346-370 (1999)). The kit is based on measuring ATP and since the ATP level is constant between cells this can be used to evaluate the number of cells. The cells were grown on 24 well plates at 1000 cells/well. Treatment with hormone was in phenol red-free medium supplemented with 10% DCC treated fetal calf serum. After 5 days hormone treatment the medium was removed and 200 μl 5% TCA was added to the well followed by 1.8 ml 80 mM Tris-acetate 2 mM EDTA pH 7.8. 20 μl of this mixture was removed to 140 μl of the Tris-acetate buffer. Luciferase activity was measured using a Berthold luminometer.

All-trans Retinoic Acid Inhibits 17β-Estradiol-Dependent Down Regulation-of HES-1 Expression in MCF-7 Cells

MCF-7 cells were grown on 150 mm plates to 70% confluency in DME medium supplemented with 10% FBS. The medium was then changed to phenolAred free DME supplemented with DCC treated FBS to which 17β estradiol 0.1 and 1 nM respectively and 1 μM of all-trans retinoic acid was added individually or in combination. The plates were then incubated for 3 days in a humidified incubator at 37° C. and 5% CO₂. Nuclear extract was produced according to Schreiber et al (E. Schreiber et al (1989) Nucleic Acids Res 17: 6419). 50 μg of this nuclear extract was then run on SDS-PAGE and blotted over to nitrocellulose membrane Hybond C super (Amersham). HES-1 protein was then detected using HES-1 antibody.

1. Use of HES-1 in the preparation of a medicament for the treatment of cancer.

2. Use according to claim 1 wherein HES-1 is in the form of a protein or polypeptide including synthetic or artificial homologues of the wild-type protein and variants of the wild-type or native protein having similar characteristics to the wild-type protein; or the corresponding nucleotide for such proteins or polypeptides.

3. Use of an inhibitor of ER activity in the preparation of an medicament for the reduction of cancer cell proliferation.

4. Use according to claim 3 wherein the ER is ERα or ERβ.

5. Use according to claim 3 or 4 wherein the inhibitor is an artiestrogen.

6. Use according to claim 5 wherein the antiestrogen is any one of 4OH tamoxifen, raloxifen or ICI 182 780 and wherein the cancer is colon or lung cancer.

7. Use according to any preceding claim wherein the HES-1 gene is upregulated.

8. Use according to claim 1 or 2 wherein there is a reduction of cancer cell proliferation.

9. A method of reducing the proliferation of cancer cells in vitro comprising increasing the level of HES-1 in those cells.

10. A method according to claim 9 wherein the HES-1 level is increased by upregulation of the HES-1 gene.

11. A method according to claim 9 or 10, wherein the HES-1 level is increased by the introduction of HES-1 protein into those cells.

12. A method according to claim 9, 10 or 11, wherein the HES-1 level is increased by we introduction of exogenous HES-1 expression in the cells by gene therapy.

13. A method according to any one of claims 9 to 12 wherein HES-1 is in the form of a protein or polypeptide including synthetic of artificial homologues of the wild-type protein and variants of the wild-type or native protein having similar characterizes to the wild-type protein; or the corresponding nucleotide for such proteins or polypeptides.

14. A method of enchancing the effect of HES-1 on the reduction of cancer cell proliferation in vitro by expression of an engineered HES-1 which exhibits improved characteristics compared to native (wild type) HES-1.

15. A method according to claim 14, wherein the engineered HES-1 is expressed by a nucleotide which replaces a native HES-1 nucleotide or which complements that native HES-1 nucleotide sequence.

16. A method according to any one of claims 9 to 15, wherein the effect of HES-1 on cell proliferation is enhanced by an antiestrogen.

17. A method according to any one of clams 9 to 16 wherein the cancer cells are selected from breast, colon, prostate and lung cancer cells.

18. A method of monitoring cell proliferation in a subject comprising monitoring the expression of PCNA or Ki67 in a sample from the subject.

19. A method according to claim 18, wherein cell proliferation is induced by estradiol.

20. A method of monitoring the effectiveness and/or progress of cancer therapy in cancer cells in vitro comprising establishing the level of HES-1 in those cells wherein a lower level of HES-1 is indicative of an increase in cancer cell proliferation.

21. A method of screening compounds for use in cancer therapy comprising determining the effect of those compounds on the effect of HES-1 on cell proliferation aid selecting compounds which enhance the effect of HES-1 on cell proliferation.

22. A method according to claim 21 comprising contacting compounds with an HES1 nucleotide sequence or expression model.

23. A method according to claim 22, wherein the compounds are selected from compounds which function as a ligand to a nuclear receptor including the TCDD receptor (AIR).

24. A method according to claim 23, wherein the nuclear receptor is selected from the retinoic acid receptor, the vitamin D receptor, estrogen receptor and the AhR receptor.

25. Use of an HES-1 nucleotide sequence or part thereof, in the preparation of a medicament for gene therapy of cancer.

26. Use according to claim 25, wherein the nucleotide sequence comprises the entire HES-1 gene.

27. Use according to claim 25 or 26, wherein the nucleotide sequence encodes a functional portion of the HES-1 protein.

28. Use according to any one of claims 25 to 27, wherein the nucleotide sequence is supplied to the cells in a vector.

29. Use according to claim 28, wherein the vector is a plasmid or virus.

30. Use according to claim 29, wherein the HES-1 nucleotide sequence or a portion thereof is expressed in the cells whereby cell proliferation is reduced.

31. Use according to any one of claims 25 to 30, wherein the HES-1 nucleotide sequence is supplied in combination with another anti-cancer therapy.

32. A pharmaceutical preparation comprising HES-1 protein or corresponding nucleotide, either native or synthetic or in any pharmacologically effective variation thereof together with a pharmaceutically acceptable carrier.

33. A pharmaceutical preparation comprising an inhibitor to the histone deacetylase regulated by HES-1.

34. A method of reducing the proliferation of cancer cells comprising increasing the levels of HES-1 in those cells.

35. A method according to claim 34, wherein HES-1 levels a increased by upregulation of the HES-1 gene.

36. A method according to claim 34 or 35 wherein HES-1 levels are increased by the introduction of HES-1 protein into those cells.

37. A method according to claim 34, 35 or 36 wherein HES-1 levels are increased by the introduction of exogenous HES-1 expression in the cells by gene therapy.

38. A method of enhancing the effect of HES-1 on the reduction of cancer cell proliferation by expression of an engineered HES-1 which exhibits improved characteristics compared to native (wild type) HES-1.

39. A method according to claim 38, wherein the engineered HES-1 is expressed by a nucleotide which replaces a native HES-1 nucleotide or which complements that native HES-1 nucleotide sequence.

40. A method according to any one of claims 34 to 39, wherein the effect of HES-1 on cell proliferation is enhanced by an antiestrogen.

41. A method according to any one of claims 34 to 40 wherein the cancer cells are selected from breast, colon, prostate and lung cancer cells.

42. A method of monitoring the effectiveness and/or progress of cancer therapy in cancer cells comprising establishing the level of HES-1 in those cells wherein a lower level of HES-1 is indicative of an increase in cancer cell proliferation.

43. A method of gene therapy comprising supplying to cells a HES-1 nucleotide sequence or part thereof.

44. A method according to claim 43, wherein the nucleotide sequence comprises the entire HES-1 gene.

45. A method according to claim 43 or 44, wherein the nucleotide sequence encodes a functional portion of the HES-1 gene.

46. A method according to any one of claims 43 to 45, wherein the nucleotide sequence is supplied to the cells in a vector.

47. A method according to claim 46, wherein the vectors a plasmid or virus.

48. A method according to claim 47, wherein the HES-1 nucleotide sequence or a portion thereof is expressed in the cells whereby cell proliferation is reduced.

49. A method according to any one of claims 43 to 48, wherein the HES-1 nucleotide sequence is supplied in combination with another anti-cancer therapy.

50. A method according to claim 49, wherein the therapy is a drug.


SEQUENCE LISTING (4 sequences)

SEQ ID NO: 1 - protein, 31 aa, Artificial Sequence
  Artificial Peptide
CMEKNSSSPVAATPASVNTTPDKPKTASEHR

SEQ ID NO: 2 - protein, 30 aa, Artificial Sequence
  Artificial Peptide
CSGTSVGPNAVSPSSGSSLTADSMWRPWRN

SEQ ID NO: 3 - DNA, 63 bp, Unknown
  Vitallogenin ERE sequence
gagcttaggtcactgtgacctgagcttaggtcactgtgacctgagctcaggtcacagtgacct

SEQ ID NO: 4 - DNA, 11 bp, Unknown
  TATA Box sequence
gggtatataat